(19) United States
(12) Patent Application Publication
(10) Pub. No.: US 2017/0039336 A1
(43) Pub. Date: Feb. 9, 2017

(54) HEALTH MAINTENANCE ADVISORY TECHNOLOGY

(71) Applicant: Microsoft Technology Licensing, LLC, Redmond, WA (US)

(72) Inventors: Hadas Bitran, Ramat Hasharon (IL); Todd Holmdahl, Redmond, WA (US); Eric Horvitz, Kirkland, WA (US); Desney S. Tan, Kirkland, WA (US); Dennis Paul Schmuland, Redmond, WA (US); Adam T. Berns, Issaquah, WA (US); Ryen William White, Woodinville, WA (US)

(73) Assignee: Microsoft Technology Licensing, LLC, Redmond, WA (US)

(21) Appl. No.: 14/971,538

(22) Filed: Dec. 16, 2015

Related U.S. Application Data
(60) Provisional application No. 62/202,118, filed on Aug. 6, 2015.

(51) Int. Cl. G06F 19/00; A61B 5/00; H04L 29/08; H04B 1/3827; H04W 4/00

(52) U.S. Cl. CPC G06F 19/3406; H04B 1/385; H04W 4/005; H04L 67/22; A61B 5/6801; A61B 5/742

(57) ABSTRACT

A computing system comprises a client computing device configured to execute a personal assistant application program. The personal assistant application program is configured to receive user data from interaction of a user with the client computing device, user interaction with additional devices, or system networked to the client computing device, to sense a user condition based on the user data received, to analyze the user condition to identify a user health issue, present, via a user interface associated with the client computing device, a suggestion for the user to treat, overcome or improve the user health issue, assess a degree to which the user has followed the suggestion, and modify subsequent suggestions to the user based on the degree to which the suggestion was followed.

### CROSS REFERENCE TO RELATED APPLICATIONS

This application claims priority to U.S. Provisional Patent Application Ser. No. 62/202,118, filed Aug. 6, 2015, the entirety of which is hereby incorporated herein by reference.

### BACKGROUND

Improving one's health is a goal that most individuals share, yet many people fall short in achieving. In our busy lives, it can be difficult to make time to visit health care providers for checkups and preventative care. And, many people have trouble following through with resolutions to eat well and exercise. It can also be difficult for people who are not feeling well to understand the causes behind their conditions. Significant challenges exist to improving the health and well-being of both individuals and entire populations, which the technological solutions described herein offer the promise of addressing.

### SUMMARY

One embodiment provides a computing system comprising a client computing device configured to execute a personal assistant application program. The personal assistant application program is configured to receive user data from interaction of a user with the client computing device, user interaction with additional devices, or system networked to the client computing device, to sense a user condition based on the user data received, to analyze the user condition to identify a user health issue, present, via a user interface associated with the client computing device, a suggestion for the user to treat, overcome or improve the user health issue, assess a degree to which the user has followed the suggestion, and modify subsequent suggestions to the user based on the degree to which the suggestion was followed.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

This disclosure will be better understood from reading the Detailed Description with reference to the attached drawing figures, wherein:

FIG. 1 shows aspects of an example computing system configured to support HMA technology;

FIGS. 2A and 2B show aspects of various example implementation environments for HMA technology;

FIGS. 3A and 3B show aspects of one example of a wearable computing device;

FIG. 4A shows aspects of another example of a wearable computing device;

FIG. 4B shows aspects of a display panel of a wearable computing device; and

FIG. 5 illustrates an example method to present, to a user of a client computing device, actionable suggestions aimed at treating a health issue of the user.

### DETAILED DESCRIPTION

This disclosure is directed to Health Maintenance Advisory (HMA) technology that formulates and presents advice to individuals and groups for the purpose of promoting good health. Before addressing the specific methodology in detail, an example infrastructure supporting the HMA technology will first be described.

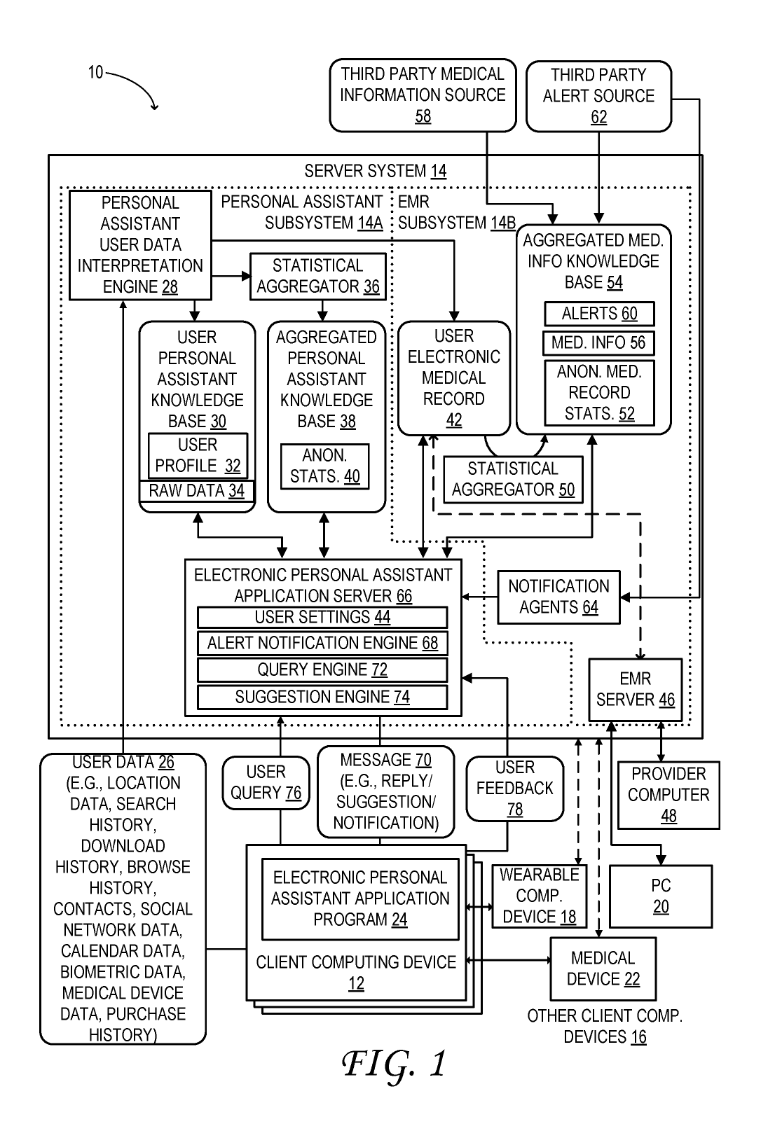

FIG. 1 shows aspects of a computing system 10 according to one embodiment of the present disclosure. As shown, computing system 10 includes a client computing device 12, which, for example, may take the form of a smart phone or tablet computing device, configured to communicate via a computer network with a server system 14. Computing system 10 may also include other client computing devices 16 configured to communicate with the server system directly through a network connection or indirectly through the client computing device 12. The other client computing devices 16 may include a wearable computing device 18, which may take the form of a wrist-mounted device or head-mounted device, a personal computer 20, which may take the form of a laptop or desktop computer, and a computerized medical device 22, such as a computerized pulse oximeter, electronic inhaler, electronic insulin delivery device, electronic blood sugar monitor, blood pressure monitor, etc. Wearable computing devices may also include sensors embedded in clothing (t-shirts, undergarments, etc.), or mounted to other body parts (e.g., a finger or ear lobe). Also envisaged are Internet of Things (IOT) devices not worn directly on the body, but arranged in physical proximity to the user. Such devices may allow measurement of biometric and other data. Examples include cameras, far-infrared thermal detectors, and under-the-mattress sleep sensors, etc. Herein, where functions of the client computing device 12 are described, it will be appreciated that any of the other client computing devices 16 may function in the same manner, unless the specific form factor of the device is mentioned explicitly.

Client computing device 12 is configured to execute an electronic personal assistant application program 24. It will be appreciated that other instances of the electronic personal assistant application program 24 may be executed on the other client computing devices 16 as well, all of which are associated with a user account on server system 14. Subject to authorization by a user, the electronic personal assistant program is configured to passively monitor various user data 26 on the client computing device 12 and other client computing devices 16, such as location data, search history, download history, browsing history, contacts, social network data, calendar data, biometric data, medical device data, purchase history, etc.

Specific examples of these various types of user data 26 will now be described. Location data may include for example, GPS coordinate data (latitude and longitude) obtained by a GPS receiver implemented on any client computing device, an identifier such as an IP address and/or Wi-Fi access point identifier that can be resolved to a generalized geographic location, a user check-in at a location via a social network program, etc. Search history may include a user's search queries entered in a search engine interface such as a browser displaying a search engine web page or a search application executed on the client computing device. The download history may include, for example, applications installed, or files downloaded from a download website, including songs, videos, games, etc. Each of these applications and files may have metadata associated with them, such as categories, genres, etc., which can be used to build user profile 32, discussed below. The browse history may include a list of websites, and particular pages within websites visited by a user using a browser executed on the client computing device. The browse history may also include in-application browsing of application specific databases, such as a shopping application that is configured to enable a user to browse a vendor's catalog. The contacts include names and contact information for individuals or organizations saved in a user contact database on client computing device 12, or retrieved from an external site, such as a social network website. The social network data may include a user's friends list, a list of social network entities “liked” by the user, check-ins made by the user at locations via a social network program, posts written by the user, etc. Biometric data may include a variety of data sensed by sensors on client computing device 12 or other client computing devices 16, such as pedometer information, heart rate and blood pressure, duration and timing of sleep cycles, body temperature, galvanic skin response, etc. Additional biometric data is discussed below in relation to the wrist-worn embodiment of the wearable computing device 18. Medical device data may include data from medical device 22. Such data may include, for example, inhaler usage data from an electronic inhaler device, blood sugar levels from an electronic blood sugar monitor, insulin pumping data from an electronic insulin pump, pulse oximetry data from an electronic pulse oximeter, etc. Purchase history may include information gleaned from an e-commerce transaction between the client computing device 12 and an e-commerce platform, regarding products purchased by a user, including product descriptions, time and date of purchase, price paid, user feedback on those purchases, etc. It will be appreciated that these specific examples are merely illustrative and that other types of user data specifically not discussed above may also be monitored.

User data 26 is transmitted from the electronic personal assistant application program 24 to the personal assistant interpretation engine 28 executed on server system 14. The personal assistant user data interpretation engine 28 performs various operations on the received user data 26, including storing copies the raw data 34 of the user data 26 in the user personal assistant knowledge base 30 (a database stored in a mass storage device of the server system 14), making inferences based upon the received user data 26 to thereby fill out a user profile 32 for the user, passing some of the user data 26 for each individual user to a statistical aggregator 36, which computes anonymized statistics 40 based on information received from all users of the server system and stores these anonymized statistics in the aggregated personal assistant knowledge base 38 (another database stored in a mass storage device of the server system 14), and passing a filtered subset of the user data to the user electronic medical record 42 based on user settings 44 in the electronic personal assistant application server 66.

As a specific example, the user profile may include inferred data from the user data 26 regarding the demographic data on the age, gender, race and ethnicity, and place of residence of the user, geographic travel history of the user, place of employment of the user, family unit of the user, family medical history, past medical history of the user, preexisting medical conditions of the user, current medications of the user, allergies of the user, surgical history, past medical screenings and procedures, past hospitalizations and visits, social history (alcohol, tobacco, and other drug use, sexual history and habits, occupation, and living conditions), health maintenance information (exercise habits, diet information, sleep data, vaccination data, therapy and counseling history), health-provider preferences, and health-benefits information.

User electronic medical records are secure electronic records stored in a database in a mass storage device associated with server system 14. Typically, data is populated within the electronic medical record for each user by a healthcare provider using provider computer 48. Provider computer 48 interacts with secure electronic medical record server 46, which in turn stores and retrieves the data in the user electronic medical record 42. The EMR server is configured to communicate over secure channels (e.g., HTTPS and TLS), and to store data in encrypted form. Further, the EMR server is configured to control access to the user electronic medical record such that only authorized healthcare providers can make entries and alter certain provider-controlled fields of the medical record. Provider controlled fields may include many of the same types of data included in the user profile, but which are confirmed with the user by the provider and entered into the medical record by the provider rather than inferred by computer algorithms; thus the accuracy and provenance of the data in the EMR is greater than the user profile 32. Specific examples of data that may be stored in the provider-controlled portion of the user electronic medical record include demographic data on the age, gender, race and ethnicity, and place of residence of the user, geographic travel history of the user, place of employment of the user, family unit of the user, family medical history, past medical history of the user, preexisting medical conditions of the user, current medications of the user, allergies of the user, surgical history, past medical screenings and procedures, past hospitalizations and visits, social history (alcohol, tobacco, and other drug use, sexual history and habits, occupation, and living conditions), health maintenance information (exercise habits, diet information, sleep data, vaccination data, therapy and counseling history), health-provider preferences, health-benefits information, and genetic profile of the user.

Other fields within the user electronic medical record are user-controlled, such that authorized persons including the patient who is the subject of the medical record can make entries in the medical record. Further, the user may adjust user settings 44 to allow the personal assistant user data interpretation engine 28 to programmatically update the user-controlled fields of the user electronic medical record with either raw data 34 or inferred data in user profile 32 derived from user data 26. In this way the medical record may be programmatically updated to include medical device data such as inhaler usage, blood sugar monitoring levels, insulin pump usage, etc., and biometric data such as heart rate and blood pressure history, sleep history, body temperature, galvanic skin response, etc.

A statistical aggregator 50 is provided to generate anonymized medical records statistics 52 based on the stored user electronic medical records of an entire user population or a predefined cohort thereof, and to store the anonymized medical record statistics in aggregated medical information knowledge base 54. In this manner, statistics may be stored for all manner of user populations. For example, a percentage of the population who live within a defined geographical region and who have been diagnosed with a certain medical condition (such as H1N1 influenza) may be identified, and data about this subset of persons may be compared to identify risk factors. The statistical aggregator may also process statistics related to steps, calories, activity level, sleep and exercise habits of an entire population, or a predefined cohort thereof, which later may be used for comparative insights on user behaviors.

Medical information 56 aggregated from third party medical information sources 58 and alerts 60 from third party alert sources 62 are also stored within the aggregated medical information knowledge base 54. In other implementations, the aggregated medical information need not be stored per se, provided it can be accessed in real time. Examples of medical information 56 includes current practices and procedures, differential diagnostic information that medical professionals use to distinguish between possible diagnoses for a given set of symptoms, descriptions of medical conditions including diseases and syndromes, and their associated symptoms, information on standardized medical screenings recommended by age and gender of the patient, information on standardized vaccination schedules recommended for children and adults, medical conditions associated with certain genetic profiles, drug information such as doses, allergens, potential interactions, etc. Examples of third party medical sources 58 include medical publishers, professional medical organizations, etc. Examples of alerts include reports from governmental and non-governmental organizations that report the occurrence of disease in particular geographic regions, including the boundaries of the geographic region, the type of disease reported, the number of persons affected, the mortality statistics associated with the affected persons, information about the incubation period, period of contiguousness for the disease, and any travel restrictions or recommended restrictions to the affected geographic region, etc. These alerts may be from a country's center for disease control, state or county health department, a company, a school district, a hospital, etc.

Alerts 60 from third party alert sources 62 may also be received by notification agents 64 within server system 14, which in turn instruct an alert notification engine 68 of the electronic personal assistant application server 66 to send a message 70 in the form of a push notification featuring the content of the alert 60 to the electronic personal assistant application program 24 executed on the client device 12, or multiple client devices running the personal assistant application program. In one specific example, the alert may be sent only to users who have recently traveled to the affected area, or who the data interpretation engine 28 infers will soon travel to the affected area, to inform the person of a disease outbreak in the particular geographic area. In another example, the alert may be sent to only to persons who have been detected by the system as being within a threshold distance of a person who has been diagnosed with a contagious disease throughout the period which the diagnosed person was contagious. Such a notification can be made while maintaining the privacy of the diagnosed individual.

In addition to push notifications for alerts 60, electronic personal assistant application server 66 also includes a query engine 72 configured to respond with messages 70 in the form of replies to a user query 76 received from the electronic personal assistant application program, and a suggestion engine 74 configured to proactively send messages 70 in the form of suggestions to the electronic personal assistant application programs based on user settings 44 and a set of programmatic suggestion rules. In one specific example, the client computing device 12 may display a query interface, such as a text box or voice prompt, and the user may type in a query or speak a query to the client computing device, such as “What could be causing this headache?” This user query 76 is sent to the query engine 72, which performs searches in each of the databases 30, 38, 42, and 52, subject to user authorizations via settings 44 to conduct searches using each of the databases. Results are returned from each database relating to causes for headaches. The user profile may indicate that the user is a “coffee drinker,” and the purchase history and location history may indicate the user visits coffee shops on average two to three times a day but has not visited a coffee shop in the past two days. The anonymized statistics may indicate that “coffee drinkers” report having headaches more often than the general population. The user electronic medical record may include a prior doctor visit in which the user complained of a headache after suffering from heatstroke. The aggregated medical information knowledge base may contain medical information that indicates that heatstroke is typically experienced when a user sweats profusely in extremely hot temperatures and experiences fast heartbeat. The raw data 34 from the user profile may show extremely hot ambient temperatures but may not show galvanic skin response indicative of sweating nor a pulse indicative of fast heartbeat. In this case the query engine would apply weightings that result in ranking the possible causes of the headache as (1) caffeine withdrawal, versus (2) heat exhaustion, and display this information to the user with a recommendation to seek the advice of a health care professional.

The electronic personal assistant application program may solicit user feedback 78 from the user regarding the effectiveness or appropriateness of the message 70, which may in turn be transmitted back to the electronic personal assistant application server 66, and used by machine-learning algorithms executed thereon to continually improve the weightings and logic by which the electronic personal assistant application server 66 makes decisions regarding the content to send to the client computing device in message 70. Continuing with the above example, if the user's headache was in fact caused by caffeine withdrawal, as diagnosed during a visit to a healthcare professional, the user might enter feedback indicating the first displayed search result was correct, and that information could then be passed to the query engine 72 as a confirmed result for machine learning algorithms that strengthen the weightings upon which the ranking was based when such confirmations are received.

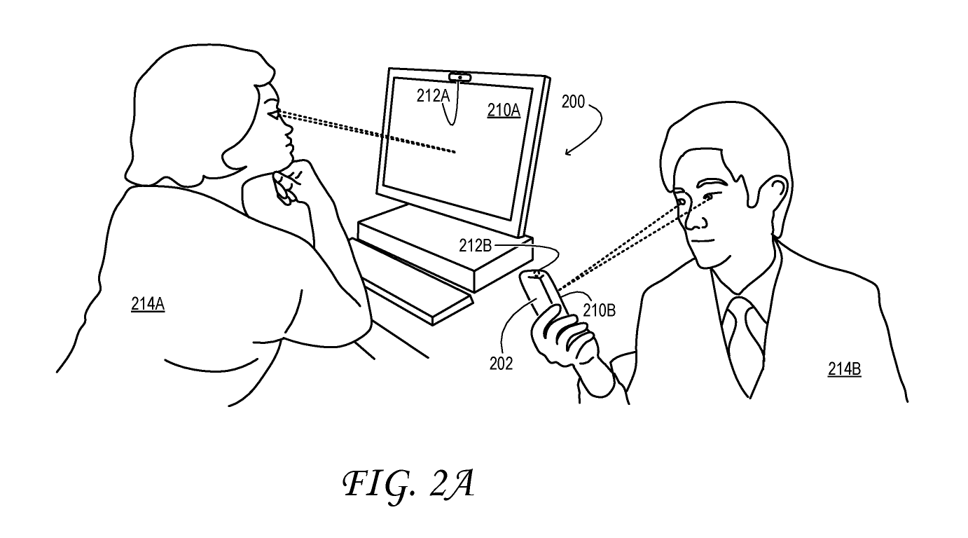

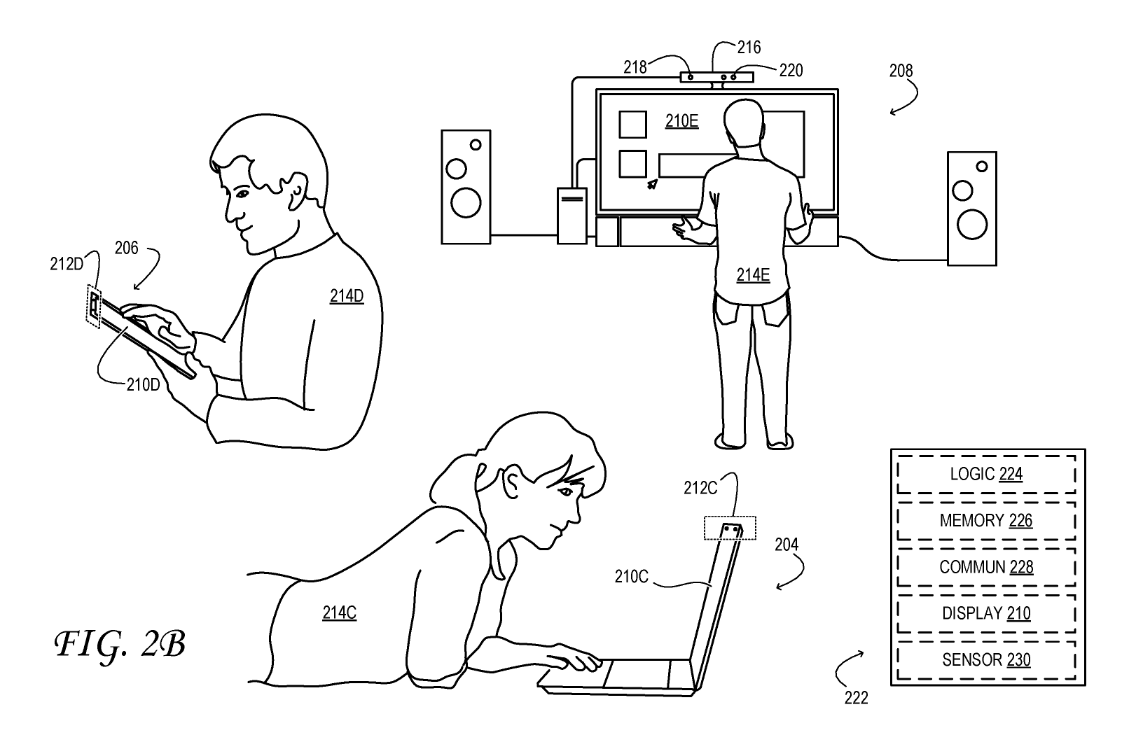

FIGS. 2A and 2B show aspects of various example client computing devices serving as client-side implementation environments for HMA technology. FIG. 2A shows a personal computer in the form of desktop computer 200. FIG. 2A also shows a client computing device in the form of smartphone 202.

Other personal computers and client computing devices are shown in FIG. 2B. This drawing shows laptop computer 204, tablet computer 206, and home-entertainment system 208. The illustrated desktop, laptop, smartphone, and tablet computer systems each include a display 210, and may also include an integrated vision system 212 configured to image the user's face, track the user's gaze or otherwise sense a facial or ocular condition of the user 214. Each vision system may include at least one camera. The home-entertainment system includes a large-format display 210E and high-fidelity vision system 216 for user face or posture detection. The high-fidelity vision system may include a color camera 218, a time-of-flight depth-sensing camera 220, and an associated infrared illuminator.

Each of the above personal computers and client computing devices may share at least some of the features of compute system 222, also shown in FIG. 2B. The compute system includes a logic machine 224 operatively coupled to a computer-memory machine 226, to display 210, to communication machine 228, and to one or more sensors 230. These and other aspects of compute system 222 will be described hereinafter.

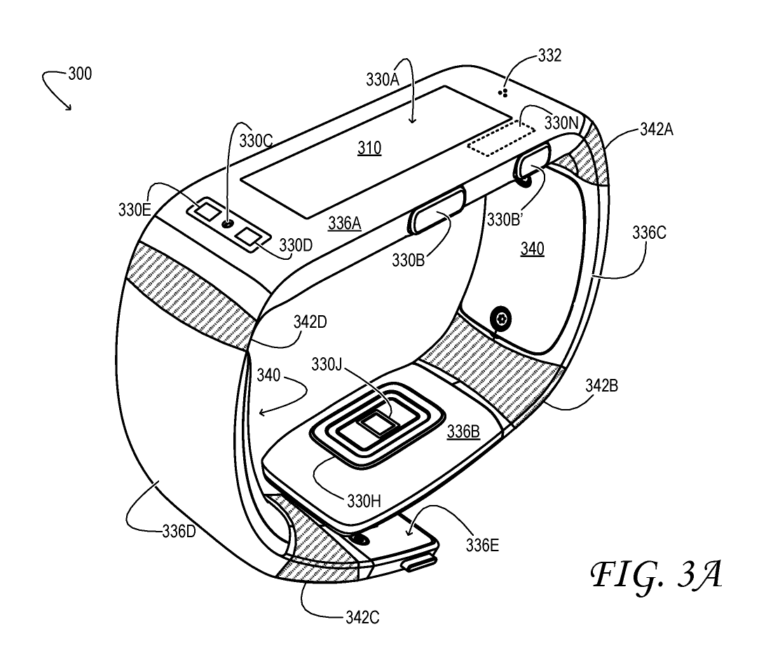

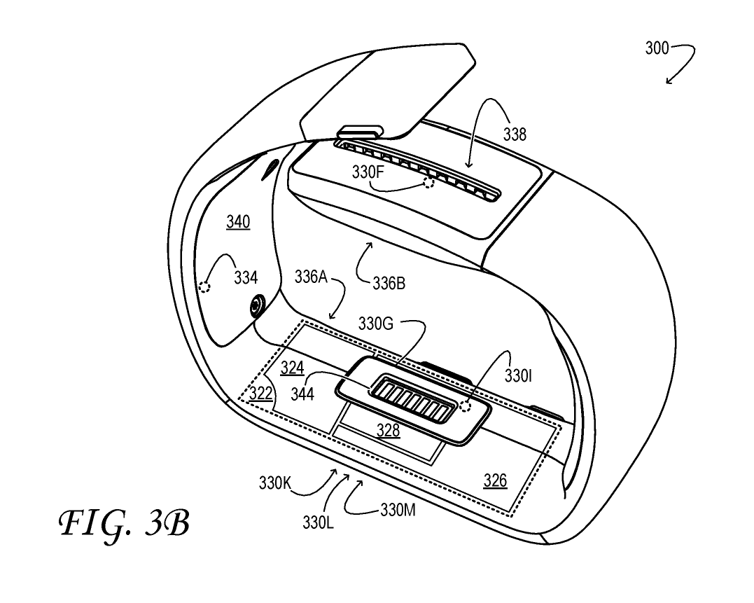

FIGS. 3A and 3B show one example of a wearable computing device configured to support HMA technology. The illustrated device takes the form of a composite band 300. In one implementation, a closure mechanism enables facile attachment and separation of the ends of the composite band, so that the band can be closed into a loop and worn on the wrist. In other implementations, the device may be fabricated as a continuous loop resilient enough to be pulled over the hand and still conform to the wrist. Alternatively, the device may have an open-bracelet form factor in which ends of the band are not fastened to one another. In still other implementations, wearable electronic devices of a more elongate band shape may be worn around the wearer's bicep, waist, chest, ankle, leg, head, or other body part.

As shown in the drawings, composite band 300 may include various functional electronic components: a compute system 322, display 310, loudspeaker 332, haptic motor 334, communication machine 328, and various sensors 330. In the illustrated implementation, functional electronic components are integrated into the several rigid segments of the band—viz., display-carrier module 336A, pillow 336B, energy-storage compartments 336C and 336D, and buckle 336E. In the illustrated conformation of composite band 300, one end of the band overlaps the other end. Buckle 336E is arranged at the overlapping end of the composite band, and receiving slot 338 is arranged at the overlapped end.

The functional electronic components of wearable composite band 300 draw power from one or more energy-storage components 340. A battery—e.g., a lithium ion battery—is one type of energy-storage electronic component. Alternative examples include super- and ultra-capacitors. To provide adequate storage capacity with minimal rigid bulk, a plurality of discrete, separated energy-storage components may be used. These may be arranged in energy-storage compartments 336C and 336D, or in any of the rigid segments of composite band 300. Electrical connections between the energy-storage components and the functional electronic components are routed through flexible segments 342.

In general, energy-storage components 340 may be replaceable and/or rechargeable. In some examples, recharge power may be provided through a universal serial bus (USB) port 344, which includes the plated contacts and a magnetic latch to releasably secure a complementary USB connector. In other examples, the energy-storage components may be recharged by wireless inductive or ambient-light charging.

In composite band 300, compute system 322 is housed in display-carrier module 336A and situated below display 310. The compute system is operatively coupled to display 310, loudspeaker 332, communication machine 328, and to the various sensors 330. The compute system includes a computer memory machine 326 to hold data and instructions, and a logic machine 324 to execute the instructions.

Display 310 may be any type of display, such as a thin, low-power light emitting diode (LED) array or a liquid-crystal display (LCD) array. Quantum-dot display technology may also be used. Suitable LED arrays include organic LED (OLED) or active matrix OLED arrays, among others. An LCD array may be actively backlit. However, some types of LCD arrays—e.g., a liquid crystal on silicon, LCOS array—may be front-lit via ambient light. Although the drawings show a substantially flat display surface, this aspect is by no means necessary, for curved display surfaces may also be used. In some use scenarios, composite band 300 may be worn with display 310 on the front of the wearer's wrist, like a conventional wristwatch.

Communication machine 328 may include any appropriate wired or wireless communications componentry. In FIGS. 3A and 3B, the communications facility includes the USB port 344, which may be used for exchanging data between composite band 300 and other computer systems, as well as providing recharge power. The communication facility may further include two-way Bluetooth, Wi-Fi, cellular, near-field communication, and/or other radios. In some implementations, the communication facility may include an additional transceiver for optical, line-of-sight (e.g., infrared) communication.

In composite band 300, touch-screen sensor 330A is coupled to display 310 and configured to receive touch input from the wearer. In general, the touch sensor may be resistive, capacitive, or optically based. Push-button sensors (e.g., microswitches) may be used to detect the state of push buttons 330B and 330B′, which may include rockers. Input from the push-button sensors may be used to enact a home-key or on-off feature, control audio volume, microphone, etc.

FIGS. 3A and 3B show various other sensors 330 of composite band 300. Such sensors include microphone 330C, visible-light sensor 330D, ultraviolet sensor 330E, and ambient-temperature sensor 330F. The microphone provides input to compute system 322 that may be used to measure the ambient sound level or receive voice commands from the wearer. Input from the visible-light sensor, ultraviolet sensor, and ambient-temperature sensor may be used to assess aspects of the wearer's environment.

A client computing device may include one or more biometric sensors configured to sense a condition of the user of the device. For instance, FIGS. 3A and 3B show a pair of contact sensors—charging contact sensor 330G arranged on display-carrier module 336A, and pillow contact sensor 330H arranged on pillow 336B. The contact sensors may include independent or cooperating sensor elements, to provide a plurality of sensory functions. For example, the contact sensors may provide an electrical resistance and/or capacitance sensory function responsive to the electrical resistance and/or capacitance of the wearer's skin. To this end, the two contact sensors may be configured as a galvanic skin-response sensor, for example. In the illustrated configuration, the separation between the two contact sensors provides a relatively long electrical path length, for more accurate measurement of skin resistance. In some examples, a contact sensor may also provide measurement of the wearer's skin temperature. In the illustrated configuration, a skin temperature sensor 330I in the form a thermistor is integrated into charging contact sensor 330G, which provides direct thermal conductive path to the skin. Output from ambient-temperature sensor 330F and skin temperature sensor 330I may be applied differentially to estimate of the heat flux from the wearer's body. This metric can be used to improve the accuracy of pedometer-based calorie counting, for example. In addition to the contact-based skin sensors described above, various types of non-contact skin sensors may also be included.

Arranged inside pillow contact sensor 330H in the illustrated configuration is an optical pulse-rate sensor 330J. The optical pulse-rate sensor may include a narrow-band (e.g., green) LED emitter and matched photodiode to detect pulsating blood flow through the capillaries of the skin, and thereby provide a measurement of the wearer's pulse rate. In some implementations, the optical pulse-rate sensor may also be configured to sense other aspects of the user's circulatory condition. The steady-state or low-pass filtered output of the photodiode may be report on the extent of capillary blood flow (i.e., vasodilation as opposed to vasoconstriction, or pallor) of the wearer. In combination with other sensory input, the optical pulse-rate sensor may be configured to estimate the wearer's blood pressure. By incorporating an LED emitter of a different wavelength band, the sensor may be configured to estimate the wearer's blood oxygenation level. In the illustrated configuration, optical pulse-rate sensor 330J and display 310 are arranged on opposite sides of the device as worn. The pulse-rate sensor alternatively could be positioned directly behind the display for ease of engineering.

Composite band 300 may also include inertial motion sensing componentry, such as an accelerometer 330K, gyroscope 330L, and magnetometer 330M. In some configurations, these components may be integrated into an inertial-measurement unit (IMU). The accelerometer and gyroscope may furnish inertial data along three orthogonal axes as well as rotational data about the three axes, for a combined six degrees of freedom. This sensory data can be used to provide a pedometer/calorie-counting function, for example. Data from the accelerometer and gyroscope may be combined with geomagnetic data from the magnetometer to further define the inertial and rotational data in terms of geographic orientation.

Composite band 300 may also include a global positioning system (GPS) receiver 330N for determining the wearer's geographic location and/or velocity. In some configurations, the antenna of the GPS receiver may be relatively flexible and extend into flexible segment 342A.

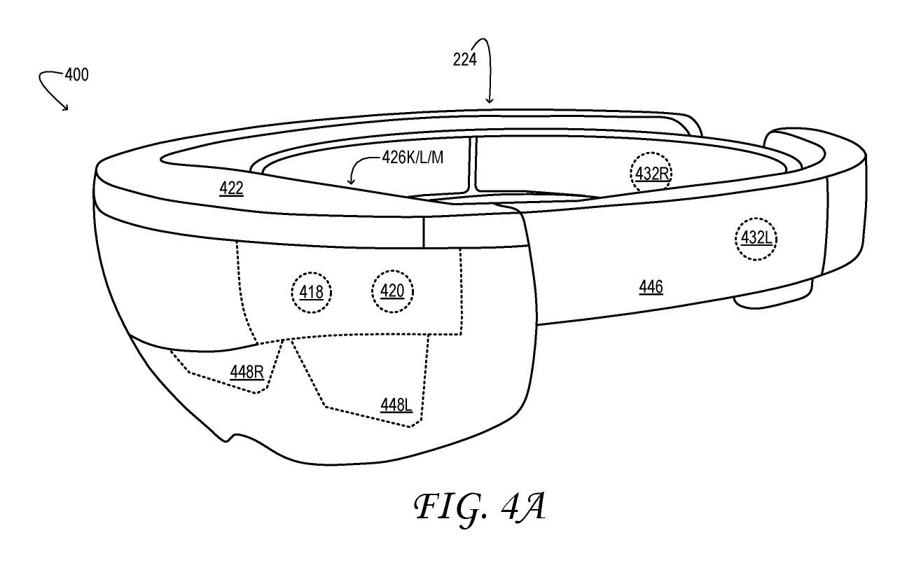

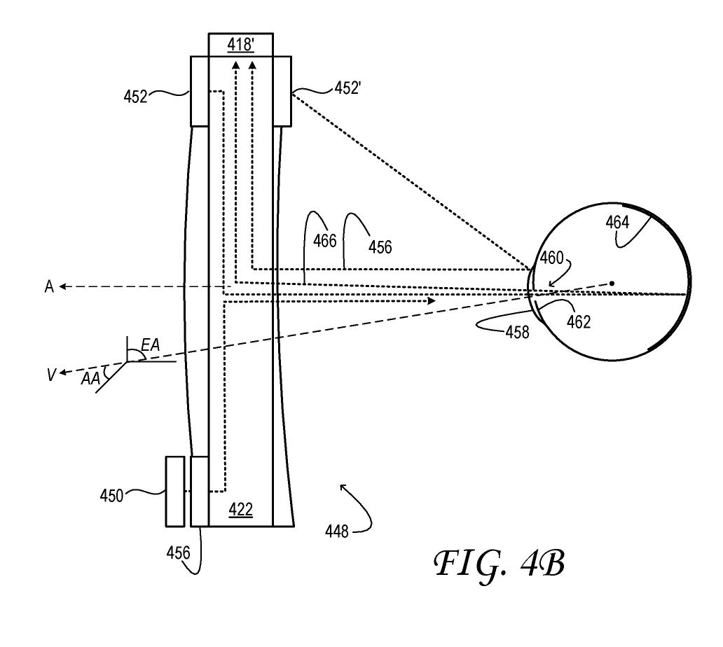

FIG. 4A shows aspects of an example head-mounted display (HMD) 400 to be worn and used by a wearer. The illustrated display system includes a frame 446. The frame supports stereoscopic, see-through display componentry, which is positioned close to the wearer's eyes. HMD 400 may be used in augmented-reality applications, where real-world imagery is admixed with virtual display imagery.

HMD 400 includes separate right and left display panels, 448R and 448L, which may be wholly or partly transparent from the perspective of the wearer, to give the wearer a clear view of his or her surroundings. Compute system 422 is operatively coupled to the display panels and to other display-system componentry. The compute system includes logic and associated computer memory configured to provide image signal to the display panels, to receive sensory signal, and to enact various control processes described herein. HMD 400 may include an accelerometer 426K, gyroscope 426L, and magnetometer 426M, stereo loudspeakers 432R and 432L, a color camera 418, and a time-of-flight depth camera 420.

FIG. 4B shows selected aspects of right or left display panel 448 (448R, 448L) in one, non-limiting embodiment. The display panel includes a backlight 450 and a liquid-crystal display (LCD) type microdisplay 410. The backlight may include an ensemble of light-emitting diodes (LEDs)—e.g., white LEDs or a distribution of red, green, and blue LEDs. The backlight may be configured to direct its emission through the LCD microdisplay, which forms a display image based on control signals from compute system 422. The LCD microdisplay may include numerous, individually addressable pixels arranged on a rectangular grid or other geometry. In some embodiments, pixels transmitting red light may be juxtaposed to pixels transmitting green and blue light, so that the LCD microdisplay forms a color image. In other embodiments, a reflective liquid-crystal-on-silicon (LCOS) microdisplay or a digital micromirror array may be used in lieu of the LCD microdisplay of FIG. 4B. Alternatively, an active LED, holographic, or scanned-beam microdisplay may be used to form right and left display images. Although the drawings show separate right and left display panels, a single display panel extending over both eyes may be used instead.

Display panel 448 of FIG. 4B includes an eye-imaging camera 418′, an on-axis illumination source 452 and an off-axis illumination source 454. Each illumination source emits infrared (IR) or near-infrared (NIR) illumination in a high-sensitivity wavelength band of the eye-imaging camera. Each illumination source may comprise a light-emitting diode (LED), diode laser, discharge illumination source, etc. Through any suitable objective-lens system, eye-imaging camera 418′ detects light over a range of field angles, mapping such angles to corresponding pixels of a sensory pixel array. Compute system 422 may be configured to use the output from the eye-imaging camera to track the gaze axis V of the wearer, as described in further detail below.

On- and off-axis illumination serve different purposes with respect to gaze tracking. As shown in FIG. 4B, off-axis illumination can create a specular glint 456 that reflects from the cornea 458 of the wearer's eye. Off-axis illumination may also be used to illuminate the eye for a ‘dark pupil’ effect, where pupil 460 appears darker than the surrounding iris 462. By contrast, on-axis illumination from an IR or NIR source may be used to create a ‘bright pupil’ effect, where the pupil appears brighter than the surrounding iris. More specifically, IR or NIR illumination from on-axis illumination source 452 illuminates the retroreflective tissue of the retina 464 of the eye, which reflects the light back through the pupil, forming a bright image 466 of the pupil. Beam-turning optics 468 of display panel 448 enable the eye-imaging camera and the on-axis illumination source to share a common optical axis A, despite their arrangement on the periphery of the display panel.

Digital image data from eye-imaging camera 418′ may be conveyed to associated logic in compute system 422 or in a remote computer system accessible to the compute system via a network. There, the image data may be processed to resolve such features as the pupil center, pupil outline, and/or one or more specular glints 456 from the cornea. The locations of such features in the image data may be used as input parameters in a model—e.g., a polynomial model—that relates feature position to the gaze axis V. In embodiments where a gaze axis is determined for the right and left eyes, the compute system may also be configured to compute the wearer's focal point as the intersection of the right and left gaze axes. In some embodiments, an eye-imaging camera may be used to enact an iris- or retinal-scan function to determine the identity of the wearer. In this configuration, compute system 422 may be configured to analyze the gaze axis, among other output from eye-imaging camera 418′ and other sensors.

The description above should not be construed to limit the range of configurations to which this disclosure applies. Indeed, this disclosure also embraces any suitable combination or subcombination of features from the above configurations. These include systems having both wrist-worn and head-worn portions, or a wrist-worn eye tracking facility, or a system in which remotely acquired sensory data is used to control a wearable or handheld display, for example.

In general, each of the client computing devices detailed above—and others within the spirit and scope of this disclosure—will include some form of user interface hardware. ‘User interface hardware’ as used herein is any physical device component of a client computing device that provides information exchange with a user of the client computing device. Any display 210, for instance, is an example of user interface hardware. Any loudspeaker coupled to a logic machine of a client computing device is an example of user interface hardware, including a loudspeaker operatively coupled to synthetic speech componentry.

Disclosed herein are configurations that allow a user or user's agent (physician, trainer, counselor, etc.) to specify one or more ‘declarative’ health outcomes. The configurations then assess various conditions of the user and adapt subsequently, so that the outcomes are achieved. In programming, the term ‘declarative’ expresses the idea that a result (i.e., the outcome) is specified absent the particular instructions to achieve it. ‘Imperative’ programming, by contrast, specifies the particular instructions that the computer must enact. Prior to the methodology disclosed herein, the user or user's agent would specify an imperative outcome—e.g., “Take two pills every 6 hours,” or “Exercise 30 minutes 3 times a week”. Departing from the imperative rubric, the configurations herein enable the user or user's agent to specify “Get patient's blood pressure to be within 5% of 120/70,” or “Improve metabolic efficiency by 12%.” These configurations both advise the user of what is needed to achieve the outcome, and continuously sense and close the control loop, so as to dynamically adjust the suggestions provided to the user.

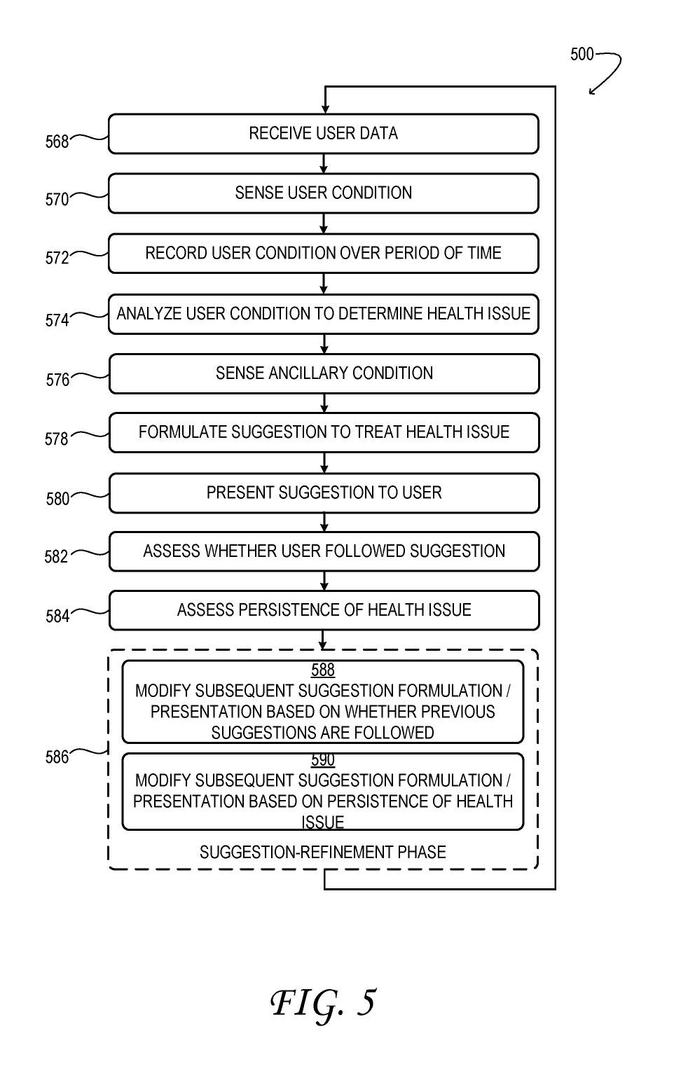

FIG. 5, accordingly, illustrates an example method 500 to identify a health issue of a user of a client computing device and present actionable suggestions to the user to treat or relieve the health issue. The term ‘health issue’ as used herein may refer to an undesirable state—physical, psychological, or behavioral. Conversely, the health issue may refer to an improving user state—e.g., a fitness plan or regimen—which the actionable suggestions support and maintain. In scenarios in which the health issue is health-related, suggestions from the client computing device may be aimed at improving the user's health. Method 500 may be enacted from a client computing device having user interface hardware, as described hereinabove, and running an electronic personal assistant application program 24, described in the context of FIG. 1. Accordingly, the client computing device may communicatively couple to an electronic personal assistant application server 66, to other components of personal assistant subsystem 14A, and to EMR subsystem 14B. It is also contemplated, however, that at least some of the aspects of method 500 may be enacted without access to the EMR subsystem, in some examples.

At 568 of method 500, user data is received by the personal assistant application server. The user data may be received from interaction of a user with the client computing device or with a system networked to the client computing device. In some implementations, the user data may include signal from a sensor integrated into the client computing device. In some implementations, the user data may be received from a server system, such as electronic personal assistant application server 66, from other components of personal assistant subsystem 14A, or from EMR subsystem 14B or from another instance of the personal assistant application program running on another client computing device. In one variant, the user data may be pushed from a networked server to the client computing device. In another variant, the user data may be pulled from the networked server by the client computing device.

At 570 a condition of the user is sensed based on the user data received. The sensed condition may be a sign or symptom of a health issue, an indication that the user is engaged in some desirable or undesirable activity, or a surrogate quantifier for the overall user health or well being of the user. In some implementations, the user condition may be sensed via signal or combination of signals from a sensor of a client computing device of the user. In one implementation, the sensor that senses the user condition may be an ocular sensor—e.g., an imaging sensor that reveals the coloration of the user's eyes and optionally tracks changes in the location of the user's gaze. In other implementations, the sensor may be a location sensor (e.g., a GPS or WiFi receiver). A location sensor may be configured to report the current location or recent path of travel of the user, for example. Other user conditions that may be sensed in this manner include pulse rate, blood oxygenation, skin temperature, ambient temperature, perspiration flux, ambient light level, ambient sound level, extent of social isolation or community (via imaging of the user's field of view and enacting face recognition or skeletal modeling), or whether the user is indoors or outdoors (via an ambient UV sensor). It will be emphasized that the above examples are not exhaustive.

In some implementations, the client computing device may support an Internet browser. Sensing the user condition may include accessing a browsing history from the Internet browser. Likewise, the client computing device may support a user calendar, which is accessed in the course of sensing the user condition. In some embodiments, the client computing device may be networked to services of a third-party financial institution and be privy to purchasing activity of the user. Accordingly, the purchasing activity of the user may be sensed at this stage of method 500. In some embodiments, the client computing device may be configured to play games or media, and/or support media streaming. Desired or undesired behaviors of the user may be sensed based on the amount of time spent in game play and/or media consumption, or on the time of day when such activities are pursued.

In these and other embodiments, the client computing device may be networked to a server system akin to server system 14 of FIG. 1. Accordingly, the client computing device may have access to the user's personal assistant knowledge base 30, user profile 32, and/or electronic medical record 42. The user condition may be sensed by accessing such data from the server system, at least in part.

In some embodiments, as indicated at optional step 572, the user condition may be recorded over a relatively long period of time (days, weeks, etc.) to establish a repeating pattern of manifestation of the user condition and/or identify exceptions in the pattern that could say something about the user's well being.

At 574, the user condition is analyzed to identify a health issue. In some scenarios, the health issue may include a medical condition of the user—i.e., a physical or psychological condition. Recurring fatigue is an example of a health issue that may be identified in this manner. In one example, fatigue may be signaled by eye redness as sensed by an ocular sensor. In another example, fatigue may be signaled by a reduction in expected saccadic movement of the eye responsive to stimuli, as sensed by eye-tracking componentry of the client computing device. In some cases, recurring fatigue may be co-morbid with a sleep disorder. Sleep disorders may be signaled by excessive arm or body motion during sleep hours, as sensed by an inertial sensor of a wrist-worn client device, for example.

In some embodiments, the health issue may include an undesired behavior of the user. Examples of undesired behaviors may include frequent trips to the bar, frequent purchase of alcohol, cigarettes, coffee, calorie-rich foods, or over-the-counter pharmaceuticals. In still other embodiments, the health issue may include absence of a desired behavior of the user. Examples of desired behaviors may include walking, running, biking, and other forms of cardiovascular exercise. Other examples of desired behaviors may include relaxing or psychologically positive recreational activities such as activities outside of the home or workplace—e.g., a trip to the movies.

It may be inferred that the psychological and perhaps cognitive health of elderly people may improve with social contact, which may be sensed by imaging an elder user's field of view and enacting face recognition and/or skeletal modeling to identify other people. Accordingly, lack of social contact over an extended period of time may be identified as a user health issue.

At 576 is sensed an ancillary condition that could potentially affect a suggestion made to the user in order to treat an identified health issue. The ancillary condition may be a condition extant in the user's environment. Examples of contemplated ancillary conditions include current GPS location, weather conditions, time of day, and events recorded on the user's calendar. In addition, any of the sensed user conditions referred to herein may also be ancillary conditions in method 500. Ancillary conditions may be used to limit or influence the suggestion to treat the user's health issue. For instance, it may be unproductive to suggest outdoor exercise to the user in the middle of the night, when it is raining, or when the user is traveling on an airplane. Further, even when a user is trying to quit smoking, it may not be advisable to remind him not to smoke prior to an important meeting.

At 578, a suggestion for the user to treat the identified health issue is formulated. Personalized suggestions can be tailored to specific conditions (rule-based or content-based filtering recommendation), or learned from crowd impact (collaborative filtering recommendation), as described hereinafter. One example suggestion formulation may include “Try decaf,” for a user experiencing poor sleep but walking towards a coffee shop at 2:00 in the afternoon. Another example suggestion may include “Time for bed,” if a user suffering from eye redness is streaming a movie at midnight. Another example suggestion may include “Call your daughter,” for an elderly user socially isolated for an extended period of time. In implementations in which ancillary conditions are sensed, the suggestion may be formulated in view of the ancillary conditions.

At 580, the formulated suggestion is presented to the user. The suggestion may be presented via user interface hardware of the client computing device. In one implementation, the user interface hardware may include a display configured to present the suggestion as text and/or mnemonic imagery presented on the display. In this and other implementations, the user interface hardware may include a loudspeaker configured to present the suggestion via synthesized speech. In these and other implementations, the suggestion may include an audible alert. In some embodiments, the mode of presentation may be modified in view of ancillary conditions. Audible presentation may be the default mode when the user is driving a car, for instance, but may be suppressed when the user is at a movie theatre. In some implementations, there may be a cap on the number of times a certain suggestion is made to a specific user before it is automatically discarded.

At 582 one or more actions are taken in order to assess whether, or to what degree, the user has followed the suggestion presented. For example, the system can determine, through continued sensing, whether the user in the above example actually went to bed early, as suggested, or ignored the recommendation and continued to stream the video. By accessing the user's purchasing activity, it can be determined whether the user continues to by cigarettes or alcohol. By interrogating an inertial-measurement unit in a wrist-worn client-computing device, it can be determined whether the user is getting more cardiovascular exercise.

At 584, follow-up sensing of the user condition is enacted in order to assess the persistence of the health issue subsequent to presentation of the suggestion. If the user with eye redness followed the suggestion to go to bed early, follow-up assessment could be used to determine whether his or her eyes are still red on the following day. Likewise, follow-up sensing enacted over a longer time scale may be used to determine to what degree suggestions to exercise or avoid calorie-rich foods have furthered the user's weight-loss goals. Access to a frequently updated health record may be used for this purpose. Follow-up sensing may also rely on access to the user's purchasing activity, browsing activity, calendar appointments, and/or location data, at least in part. It will be understood that the above list is not exhaustive.

At 586 a suggestion-refinement phase is entered, whereby one or more of (a) the formulation of future suggestions, or (b) the mode of presentation of future suggestions is modified based on the established efficacy of the suggestions. At 588 of method 500, modification is enacted pursuant to whether, or to what extent, the suggestion was followed by the user, as determined at 582. For example, some users may respond well to suggestions presented by text or mnemonic imagery, while other users may require audible, verbal prompting in order to follow suggestions reliably. This information may be gathered by trying each presentation mode individually (i.e., serially), and determining for each mode whether or not the user complied with the suggestion. Subsequently, the mode of suggestion to the same user—or to different user's of a common age group or demographic—may be altered to align with the most positive user response.

At 590, modification is enacted pursuant to the follow-up sensing at 584 (i.e., the persistence of the health issue). In many cases, for example, there may be more than one potentially appropriate remedy for a given health issue. An overweight user could be recommended to avoid trips to the snack bar, on the one hand, or to engage in cardiovascular exercise, on the other. In one example, the efficacy of each suggestion in effecting weight loss over a suitable period of time may be determined by offering each suggestion at the exclusion of the other, while concurrently accessing the user's body weight via his or her health record. If it becomes evident that one form of suggestion is more effective than the other, for a particular user, then that form may be used to assist the same or similar users in continued or future weight-loss activity. In implementations and scenarios in which the appropriate suggestion-space is multivariate, detailed statistical analysis may be conducted in order to rank competing suggestions. Diversification of suggestions may also take place in order to make sure the user is offered with fresh and interesting suggestions for greater overall impact.

It will be emphasized that suggestion-refinement phase 586, and other aspects of method 500, may take different forms depending on the embodiment being practiced. In a community environment, for example, the condition of a plurality of client-device users may be sensed and analyzed concurrently to identify a common health issue. But instead of formulating and presenting the same suggestion for all of the users, the system may formulate a first suggestion for a first user in the group, and a second, different, suggestion for a second user. Different suggestions may be formulated and presented even though the first and second users share the same health issue, and may also have other features in common (e.g., they belong to the same age group, race, or socioeconomic class, or share the same occupation or other demographic). Follow-up sensing of user condition after presentation of the first and second suggestions may be enacted in order to compare the persistence of the health issue in the first and second users. Then, the first and second suggestions may be ranked based on efficacy in treating the common health issue. When method 500 is executed afresh—on, say, a third user from the same demographic—that user may receive the suggestion previously shown to be more successful in treating the health issue. Naturally, if a large pool of users sharing common traits is available, then a statistical approach may be taken to rank competing suggestions. It will be noted that the above methods may be used in communities of significant population (e.g., a corporate workforce). In this manner, an employer may actively provide suggestions to the employees to encourage improved health, well-being, and productivity.

Numerous variations and extensions on the method of FIG. 5 are envisaged. The following additional examples illustrate the important aspect that the user condition sensed at 570 of method 500 need not be an isolated condition, but may be a composite condition derived from two or more conditions concurrently sensed.

The first additional example relates to fitness assessment and helping the user to achieve his or her fitness goals. Cardiovascular fitness of a healthy individual may be correlated to the level of caloric output that individual can sustain at a given pulse rate. To put it another way, the same level of caloric output will elicit a lower pulse-rate response from a fit individual than from an unfit individual. Traditionally, a treadmill with pulse-rate monitoring may be used to assess this type of fitness. However, composite band 300 (the client computing device illustrated in FIGS. 3A and 3B) provides the sensory components needed to enable a treadmill-like assessment, but without confining the user to an actual treadmill. The client computing device provides, in other words, a ‘virtual treadmill’ function.

As noted hereinabove, optical pulse-rate sensor 330J provides direct, real-time monitoring of the user's pulse rate. IMU accelerometer 330K and gyroscope 330L provide inertial data from which the user's caloric output may be estimated. For improved accuracy, the caloric-output calculation may reference a body model and behavior model specific to the user. Such models may be stored in the memory of the client computing device, or on the server system 14, for example. The caloric-output calculation may also reference location data, which can be obtained from GPS receiver 330N of the client computing device.

In some examples, cardiovascular fitness assessment can be enacted automatically with no purposeful effort by the user. It may be triggered whenever the user ascends a flight of stairs, runs to catch a bus, or engages in other serendipitous forms of physical exercise. Naturally, the fitness assessment may also be triggered by purposeful physical exercise.

At 570 of method 500, therefore, the sensed user condition may be the level of cardiovascular fitness assessed compositely, as described above. On behalf of a user wishing to become more fit, the system may monitor the fitness level over a period of time, and at 580, present suggestions aimed at improving the user's fitness. As mentioned previously, suggestions may be directed to reminding the user to avoid of calorie-rich foods and/or tobacco, to schedule time for exercise, to get more sleep, etc. Follow-up monitoring may be used to determine the degree to which the various suggestions are followed, and/or the degree to which followed suggestions appear to increase the user's fitness level.

The above example demonstrates fitness assessment using only a wearable device and associated services. While that aspect is advantageous in many scenarios, the present disclosure also extends to implementations in which sensory data derived from more specialized networked equipment is interpreted and used to assess fitness. In one alternative, an exercise machine such as an actual treadmill, rowing machine, elliptical machine, etc.—with biometric sensors capable of pulse rate and/or blood-pressure monitoring—may be used to perform fitness testing. Such exercise machines may be accessible to client computing device via a wireless connection such as a short-range network (e.g., BLUETOOTH or WiFi), or over the Internet to server system 14. Similarly, a networked electronic scale configured to measure body-weight scale and/or body-mass index (BMI) may measure the user's body weight and BMI and send this information to the wearable device via a similar wireless connection as the exercise machines, to further contribute to fitness assessment.

The second additional example relates generally to improving the psychological health of the user, and more specifically, to assessing the user's ability to avoid anxiety and panic. Physiological manifestations of anxiety vary among individuals, but generally include increased pulse rate, increased perspiration, increased pallor, and pupil dilation. Pulse rate, perspiration, and pallor are directly measurable via the sensory architecture of composite band 300 (vide infra). Pupil dilation is measurable via eye-imaging camera 418′ of HMD 400 (the client computing device illustrated in FIGS. 4A and 4B), or via any integrated vision system 212 or 218 (FIGS. 2A and 2B).

Though readily measurable, none of the conditions noted above is specific to anxiety. Perspiration and (as the previous example illustrates) pulse rate also correlate to caloric output. Perspiration further correlates to high ambient temperature, and pallor to low ambient temperature. Pupil dilation correlates to low ambient light levels and to sexual arousal. While none of the above sensory outputs taken alone may be a suitable indicator of anxiety level, appropriate linear combinations of such outputs, and ancillary conditions such as ambient temperature, ambient light level, etc., also measurable in real time, may provide accurate surrogates. In some embodiments, a machine learning implementation may be used to arrive at weightings of individual classifiers in the linear combination. Machine learning may follow a supervised-learning approach, in which the figure of merit (level of anxiety) is input by the user's clinician (who can modify the user's health record). In other examples, direct input from the user on the client computing device may be used to distinguish anxiety from other conditions, for example, by the computing prompting the user with a prompt such as “You seem anxious, are you?” and receiving user input indicating a yes or no answer. In some examples, a boosting algorithm may be used to build a strong composite classifier from a plurality of weak, ad-hoc classifiers. Virtually any type of sensory data from biometric or other sensors may be combined in this manner.

At 570 of method 500, therefore, the sensed user condition may be the user's anxiety level, as described above. On behalf of a user wishing to control his or her anxiety, avoid panic, etc., the system may monitor the anxiety level over a period of time, and at 580, present suggestions aimed at reducing it. Suggestions may be directed to influencing the user to avoid stressful situations and/or psychoactive substances such as alcohol, nicotine, or caffeine, to schedule time for exercise, to get more sleep, etc. Follow-up monitoring may be used to determine the degree to which the various suggestions are followed, and/or the degree to which followed suggestions appear to increase the user's anxiety level. For example, if the suggestion is to exercise or more get more sleep, the sensors within the wearable computing device may be used to determine that a user is exercising or sleeping according to the suggestion, and future suggestions may be modified based on the degree to which the suggestions are followed by the user, and also based on the degree to which the followed suggestions are effective at reducing the anxiety level, as measured through the biometric sensors discussed above.

The methods herein, which involve the observation of people in their daily lives, may and should be enacted with utmost respect for personal privacy. Accordingly, the methods presented herein are fully compatible with opt-in participation of the persons being observed. In embodiments where personal data is collected on a local computer and transmitted to a remote computer for processing, that data can be anonymized in a known manner. In other embodiments, personal data may be confined to a local computer, and only non-personal, summary data transmitted to a remote computer. Further, although various scenarios may be described herein relating to the treatment of illness in a context specific manner to the user, the information presented to the user is not intended to be medical advice, and in these scenarios the user is typically presented with a notification encouraging the user to seek the advice of a healthcare professional. The scenarios are illustrative, and chosen for ease of understanding to non-medical professionals, so that the overall methodology of this disclosure will be more clearly appreciated.

As evident from the foregoing description, the methods and processes described herein may be tied to a compute system of one or more computing devices. In particular, such methods and processes may be implemented as a computer-application program or service, an application-programming interface (API), a library, and/or other computer-program product.

Returning now to FIG. 2B, the drawing schematically shows a non-limiting embodiment of a compute system 222 that can enact one or more of the methods and processes described above. Compute system 222 is shown in simplified form. Compute system 222 may take the form of one or more personal computers, server computers, tablet computers, home-entertainment computers, network computing devices, gaming devices, mobile computing devices, mobile communication devices (e.g., smartphone), and/or other computing devices.

Compute system 222 includes a logic machine 224 and a computer memory machine 226. Compute system 222 may optionally include a display 210, input or sensory subsystem 230, communication machine 228, and/or other components not shown in the drawings.

Logic machine 224 includes one or more physical devices configured to execute instructions. For example, the logic machine may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

The logic machine may include one or more processors configured to execute software instructions. Additionally or alternatively, the logic machine may include one or more hardware or firmware logic machines configured to execute hardware or firmware instructions. Processors of the logic machine may be single-core or multi-core, and the instructions executed thereon may be configured for sequential, parallel, and/or distributed processing. Individual components of the logic machine optionally may be distributed among two or more separate devices, which may be remotely located and/or configured for coordinated processing. Aspects of the logic machine may be virtualized and executed by remotely accessible, networked computing devices configured in a cloud-computing configuration.

Computer memory machine 226 includes one or more physical devices configured to hold instructions executable by the logic machine to implement the methods and processes described herein. When such methods and processes are implemented, the state of computer memory machine 226 may be transformed—e.g., to hold different data.

Computer memory machine 226 may include removable and/or built-in devices. Computer memory machine 226 may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. Computer memory machine 226 may include volatile, nonvolatile, dynamic, static, read/write, read-only, random-access, sequential-access, location-addressable, file-addressable, and/or content-addressable devices.

It will be appreciated that computer memory machine 226 includes one or more physical devices. However, aspects of the instructions described herein alternatively may be propagated by a communication medium (e.g., an electromagnetic signal, an optical signal, etc.) that is not held by a physical device for a finite duration.

Aspects of logic machine 224 and computer memory machine 226 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of compute system 222 implemented to perform a particular function. In some cases, a module, program, or engine may be instantiated via logic machine 224 executing instructions held by computer memory machine 226. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

It will be appreciated that a “service”, as used herein, is an application program executable across multiple user sessions. A service may be available to one or more system components, programs, and/or other services. In some implementations, a service may run on one or more server-computing devices.

When included, display 210 may be used to present a visual representation of data held by computer memory machine 226. This visual representation may take the form of a graphical user interface (GUI). As the herein described methods and processes change the data held by the storage machine, and thus transform the state of the storage machine, the state of display 210 may likewise be transformed to visually represent changes in the underlying data. Display 210 may include one or more display devices utilizing virtually any type of technology. Such display devices may be combined with logic machine 224 and/or computer memory machine 226 in a shared enclosure, or such display devices may be peripheral display devices.

When included, input or sensory subsystem 230 may comprise or interface with one or more user-input devices such as a keyboard, mouse, touch screen, or game compute system. In some embodiments, the input or sensory subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity.

When included, communication machine 228 may be configured to communicatively couple compute system 222 with one or more other computing devices. Communication machine 228 may include wired and/or wireless communication devices compatible with one or more different communication protocols. As non-limiting examples, the communication machine may be configured for communication via a wireless telephone network, or a wired or wireless local- or wide-area network. In some embodiments, the communication machine may allow compute system 222 to send and/or receive messages to and/or from other devices via a network such as the Internet.

One aspect of this disclosure is a computing system comprising: a wearable client computing device configured to execute a personal assistant application program, the personal assistant application program being configured to: receive user data from interaction of a user with a biometric sensor of the client computing device or system networked to the client computing device; sense a user condition based on the user data received; analyze the user condition to identify a user health issue; present, via a user interface associated with the client computing device, a suggestion for the user to treat the user health issue; assess a degree to which the user has followed the suggestion; and modify subsequent suggestions to the user based on the degree to which the suggestion was followed.

In this aspect, the personal assistant application program may be configured to sense the user condition based on user data pushed from the system networked to the client computing device. In this aspect, the biometric sensor may be arranged in the client computing device, and the personal assistant application program may be configured to sense the user condition via signal from the biometric sensor. In this aspect, the client computing device may further include a location sensor, and the personal assistant application program may be configured to sense the user condition at least in part by accessing a travel path determined by the location sensor. In this aspect, the client computing device may be configured to execute an Internet browser, and the personal assistant application program may be configured to sense the user condition by accessing a browsing history of the Internet browser. In this aspect, the personal assistant application program may be configured to sense the user condition by accessing a user calendar. In this aspect, the computing system may be privy to purchasing activity of the user, and the personal assistant application program may be configured to sense the user condition based on the purchasing activity. In these and other implementations, the personal assistant application program may be further configured to record the user condition over a period of time to identify a repeating pattern of user behavior, and the health issue may be identified based on the user behavior. In this aspect, the health issue may include a medical condition. In this aspect, the health issue may include an undesired behavior of the user. In this aspect, the health issue may include absence of a desired behavior of the user. In this aspect, the personal assistant application program may be further configured to sense an ancillary condition in an environment of the user and to formulate the suggestion in view of the ancillary condition. In this aspect, the user interface may include a display configured to present the suggestion as text and/or imagery. In this aspect, the biometric sensor may be one of a plurality of biometric sensors of the client computing device or system networked to the client computing device, and the user condition may be a composite condition sensed via user data received from the plurality of biometric sensors.

Another aspect of this disclosure is a method enacted in a wearable client computing device configured to execute a personal assistant application program and having user interface hardware. The method comprises: sensing a user condition based on user data received from a biometric sensor; analyzing the user condition to identify a user health issue; formulating a suggestion for the user to treat or improve the user health issue; presenting the suggestion to the user via the user interface hardware; assessing a degree to which the user has followed the suggestion; and assessing persistence of the user health issue by follow-up biometric sensing of the user condition.

In this aspect, the method may further comprise modifying subsequent suggestion formulation or presentation based on the assessed persistence of the user health issue. In this aspect, the method may further comprise modifying subsequent suggestion formulation or presentation based on the degree to which the suggestion was followed. In this aspect, sensing the user condition may include executing a personal assistant application program on the client device, and said analyzing, formulating, and presenting may include receiving user data in the personal assistant application program from a system networked to the client computing device.

Another aspect of this disclosure is a method enacted in a computing system including a plurality of wearable client computing devices, each client computing device configured to execute a personal assistant application program and having user interface hardware. The method comprises: sensing a user condition of first and second users based on user data received from biometric sensors; analyzing the user condition of the first and second users to identify a health issue common to the first and second users; formulating a first suggestion for the first user and a second suggestion for the second user, to treat the common health issue; presenting the first suggestion to the first user and the second suggestion to the second user, on the client computing devices of the first and second users; comparing persistence of the common health issue in the first and second users by follow-up sensing of the user condition of the first and second users; ranking the first and second suggestions based on efficacy of treating the health issue in the first and second users; presenting the first suggestion to a third user, on the client computing device of the third user if the first suggestion is ranked higher than the second suggestion; and presenting the second suggestion to the third user, on the client computing device of the third user if the second suggestion is ranked higher than the first suggestion.

In this aspect, the first, second, and third users may share a commonality besides the common health issue, and the suggestion may be presented to all users sharing that commonality.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A computing system, comprising:
a wearable client computing device configured to execute a personal assistant application program, the personal assistant application program being configured to:
receive user data from interaction of a user with a biometric sensor of the client computing device or system networked to the client computing device;
sense a user condition based on the user data received;
analyze the user condition to identify a user health issue;
present, via a user interface associated with the client computing device, a suggestion for the user to treat the user health issue;
assess a degree to which the user has followed the suggestion; and
modify subsequent suggestions to the user based on the degree to which the suggestion was followed.

2. The computing system of claim 1 wherein the personal assistant application program is configured to sense the user condition based on user data pushed from the system networked to the client computing device.

3. The computing system of claim 1 wherein the biometric sensor is arranged in the client computing device, and wherein the personal assistant application program is configured to sense the user condition via signal from the biometric sensor.

4. The computing system of claim 3 wherein the client computing device further includes a location sensor, and wherein the personal assistant application program is configured to sense the user condition at least in part by accessing a travel path determined by the location sensor.

5. The computing system of claim 1 wherein the client computing device is configured to execute an Internet browser, and wherein the personal assistant application program is configured to sense the user condition by accessing a browsing history of the Internet browser.

6. The computing system of claim 1 wherein the personal assistant application program is configured to sense the user condition by accessing a user calendar.

7. The computing system of claim 1 wherein the computing system is privy to purchasing activity of the user, and the personal assistant application program is configured to sense the user condition based on the purchasing activity.

8. The computing system of claim 1 wherein the personal assistant application program is further configured to record the user condition over a period of time to identify a repeating pattern of user behavior, and wherein the health issue is identified based on the user behavior.

9. The computing system of claim 1 wherein the health issue includes a medical condition.

10. The computing system of claim 1 wherein the health issue includes an undesired behavior of the user.

11. The computing system of claim 1 wherein the health issue includes absence of a desired behavior of the user.

12. The computing system of claim 1 wherein the personal assistant application program is further configured to sense an ancillary condition in an environment of the user and to formulate the suggestion in view of the ancillary condition.

13. The computing system of claim 1 wherein the user interface includes a display configured to present the suggestion as text and/or imagery.

14. The computing system of claim 1 wherein the biometric sensor is one of a plurality of biometric sensors of the client computing device or system networked to the client computing device, and wherein the user condition is a composite condition sensed via user data received from the plurality of biometric sensors.

15. Enacted in a wearable client computing device configured to execute a personal assistant application program and having user interface hardware, a method comprising:
sensing a user condition based on user data received from a biometric sensor;
analyzing the user condition to identify a user health issue;
formulating a suggestion for the user to treat or improve the user health issue;
presenting the suggestion to the user via the user interface hardware;
assessing a degree to which the user has followed the suggestion; and
assessing persistence of the user health issue by follow-up biometric sensing of the user condition.

16. The method of claim 15 further comprising modifying subsequent suggestion formulation or presentation based on the assessed persistence of the user health issue.

17. The method of claim 15 further comprising modifying subsequent suggestion formulation or presentation based on the degree to which the suggestion was followed.

18. The method of claim 15 wherein sensing the user condition includes executing a personal assistant application program on the client device, and wherein said analyzing, formulating, and presenting include receiving user data in the personal assistant application program from a system networked to the client computing device.

19. Enacted in a computing system including a plurality of wearable client computing devices, each client computing device configured to execute a personal assistant application program and having user interface hardware, a method comprising:
sensing a user condition of first and second users based on user data received from biometric sensors;
analyzing the user condition of the first and second users to identify a health issue common to the first and second users;
formulating a first suggestion for the first user and a second suggestion for the second user, to treat the common health issue;
presenting the first suggestion to the first user and the second suggestion to the second user, on the client computing devices of the first and second users;
comparing persistence of the common health issue in the first and second users by follow-up sensing of the user condition of the first and second users;
ranking the first and second suggestions based on efficacy of treating the health issue in the first and second users;
presenting the first suggestion to a third user, on the client computing device of the third user, if the first suggestion is ranked higher than the second suggestion; and
presenting the second suggestion to the third user, on the client computing device of the third user, if the second suggestion is ranked higher than the first suggestion.

20. The method of claim 19 wherein the first, second, and third users share a commonality besides the common health issue, and wherein the suggestion is presented to all users sharing that commonality.
